(19) United States
(12) Patent Application Publication
(10) Pub. No.: US 2014/0037607 A1
(43) Pub. Date: Feb. 6, 2014

(54) CULTURE MEDIUM AND METHOD FOR OBTAINING A POPULATION OF TOLEROGENIC DENDRITIC CELLS

(71) Applicants: Hakim Hocini, Creteil (FR); Renato Monteiro, Paris (FR); Hicham Bouhlal, Amiens (FR); Agnes Lehuen, Paris (FR); Julien Diana, Paris (FR)

(72) Inventors: Hakim Hocini, Creteil (FR); Renato Monteiro, Paris (FR); Hicham Bouhlal, Amiens (FR); Agnes Lehuen, Paris (FR); Julien Diana, Paris (FR)

(73) Assignee: Institut National de la Sante et de la Recherche Medicale(INSERM), Paris (FR)

(21) Appl. No.: 14/009,999

(22) PCT Filed: Apr. 5, 2012
(86) PCT No.: PCT/EP12/56365

§ 371 Date: Oct. 4, 2013

(30) Foreign Application Priority Data
Apr. 8, 2011 (EP) 11305412.6

(51) Int. Cl. C12N 5/0784

(52) U.S. Cl. CPC C12N 5/064
USPC 424/93.71; 530/387.1; 435/405; 435/404; 435/372

(57) ABSTRACT

The present invention relates to a culture medium suitable for inducing dendritic cell differentiation comprising an effective amount of secretory immunoglobulins A (SIgA) and also to a method for obtaining a population of tolerogenic dendritic cells from cells, in particular monocytes. The present invention relates to uses of tolerogenic dendritic cells thus obtained in therapy and in induction of transplant tolerance.

### FIELD OF THE INVENTION

The present invention relates to a culture medium and to a method for obtaining a population of tolerogenic dendritic cells from cells, in particular monocytes. The present invention also relates to uses of tolerogenic dendritic cells thus obtained in therapy and in induction of transplant tolerance.

### BACKGROUND OF THE INVENTION

Diseases of the immune cells encompass broad categories of disorders including inflammation (e.g. asthma) and autoimmune diseases (e.g. rheumatoid arthritis, type I diabetes, psoriasis and multiple sclerosis). Dendritic cell-based immune therapies that exploit natural mechanisms of immune modulation are a promising method for treating auto-immune disorders. It may be used as a sole treatment or as an addition to other types of therapies such as in combination with other immunosuppressive drugs. The strategy is based on ex vivo manipulation and reintroduction of cellular products to circumvent auto-immune disorders for the purpose of inducing antigen-specific tolerance. Thus, the ultimate goal of such dendritic cell-based immune therapies is the induction of tolerance in the form of delivering an inhibitory signal to effector cells in vivo and recent advances have focused on induction and expansion of regulatory T cells. For example, patients with autoimmune diseases may benefit from treatment based on such dendritic cell-based vaccination strategies.

Induction of antigen-specific immune responses requires the engagement of professional antigen presenting cells (APC) expressing Major Histocompatibility Complex (MHC) molecules as well as membrane-bound and secreted co-stimulatory molecules. Dendritic cells (DCs) are the professional APC of the immune system. In peripheral tissues, DCs are found in an immature stage specialized in the capture and processing antigen. In response to antigens stimulation DC undergo a process of maturation into antigen-presenting cells able to activate both CD4+ and CD8+ T cells and polarize naïve cells into Th1, Th2, Treg or TH17. Given appropriate signals and in the absence of costimulatory markers, DCs induce peripheral tolerance as well observed in the mucosal environment where DCs control immune response even if they are in permanent interaction with the luminal commensal microbiota antigens. Alterations of DCs homeostasis have been implicated in various human autoimmune diseases such as multiple sclerosis, type 1 diabetes and systemic lupus erythematosus. The stimulatory or inhibitory capacity of DC is achieved through signals from the micro-environment such as antigens, cellular interactions and soluble factors. Thus, DCs with their dual-functions in the induction of immunity and tolerance are the main regulators of the immune system.

The induction of T cell immunity or tolerance by DCs crucially depends on the level of membrane co-stimulatory molecules such as CD40, CD80, CD83 and CD86 expressed on DC membrane surface as well as on the secretion of soluble factors such as cytokines IL-12p70, IL-23, IFN and IL-10.

However accumulating evidence suggests that there are a number of characteristic features that are critical for the function of tolerogenic DCs including:

(1) reduced expression of T cell co-stimulatory molecules (CD80, CD83, CD86),

(2) secretion of IL-10 and TGF-b cytokines

(3) down-regulation of IL-12 (p35/p40) and IL-23 (p23/p40)

4) Induction of CD4 CD25 Foxp3 T cells.

It is now well established that immunoglobulins (Igs) play an essential role in the homeostasis of the immune system. Igs are thus critical in preventing inflammatory and auto-immune diseases (18). For instance, therapeutic intervention by intravenous immunoglobulins (IVIG), that are essentially composed of a pool of IgG from healthy patients, decreases the frequency of auto-immune manifestations (18). Serum monomeric IgA can induce both the activation or the inhibition of the immune system depending of how their specific cell surface receptors are engaged (9, 19). IgA receptors (IgA-R) family comprises five members including the myeloid IgA Fc receptor (FcαRI or CD89), the Fcα/μR, the polymeric Igs receptor (pIgR) and two alternatives IgA-R: asialoglycoprotein receptor (ASGP-R) and the transferrin receptor (TfR/CD71). FcαRI is the only IgA-R which binds exclusively IgA. All other IgA-R are able to bind IgA and other Igs (Fcα/μ-R and pIg-R) or IgA and other non-Ig-related ligands (ex: ASGP-R, TfR) (15). In humans, the function of IgA-induced effectors is mainly dependent on the triggering of CD89/FcαRI expressed on myeloid cells. Recently, it has been demonstrated that monomeric IgA binding to FcαRI induces cell inhibition, which involves the immunoreceptor tyrosine-based activation motif (ITAM) present in its associated signaling unit, the γ chain (19). Furthermore, patients presenting selective IgA deficiency, the most common primary immunodeficiency worldwide, show an increased incidence of allergies and auto-immune disorders (8).

In contrast to monomeric IgA, the role of secretory IgA (SIgA) as modulator of the DCs functions remains neglected. Indeed, despite the high amount of polymeric IgA that is synthetized in mucosa before their secretion across the mucosa epithelium, there are few studies that address its function in relationship to immune regulation of both tolerance and activation. SIgA is the most abundant class of immunoglobulin synthesized in humans, with 40 to 60 mg per kg of body weight produced daily (3, 5). Most of these Igs are natural Abs produced in the absence of deliberate immunization (13, 20, 24). The SIgA is part of the first-line specific immune barrier in mucosa through a mechanism called immune exclusion. In vivo, exogenously delivered SIgA is able to enter into multiple Peyer's patch lining the intestine where it is transported by intestinal M cells before interacting with DCs in the subepithelial dome region, prior to their internalization (6, 7, 21). Furthermore, when used as a carrier for antigens in oral immunization, SIgA induces mucosal and systemic responses associated with production of anti-inflammatory cytokines (4).

Circulating monocytes are an excellent source for DCs generation in vitro but their contribution to DC homeostasis in vivo is still unclear.

Monocytes can give rise to lymphoid organ DCs in vivo during inflammation (17, 22). Monocytes have been shown to contribute to DC homeostasis in the intestine in the noninflamed setting (25, 26). Consistent with this hypothesis, a study in the rat has shown that monocytes give rise to tissue migratory DCs suggesting that monocytes could contribute to the lamina propria DCs (27). These studies lead to the hypothesis that monocytes represent a major contributor of the intestinal DC pool in the steady state.

DCs at mucosa that originating from myeloid monocytes are in continuous contact with polymeric IgA during their differentiation steps. Indeed, mucosal tissues contain a preponderance (70-90%) of IgA+ plasma cells, which in the normal human gut represent at least 80% of all plasma cells in the body (14). Especially, in the mucosal environment, where DCs are critical for the control of inflammation and the maintenance of immune tolerance even if they are in permanent interaction with the luminal commensal microbiota antigen (11). Indeed, DCs express Pattern Recognition Receptors (PRRs) such as the well-known Toll-like receptors (TLR), which recognize a plethora of bacterial, viral and fungal conserved motifs known as Pathogen-Associated Molecular Patterns (PAMPs) (10, 12). The mucosal immune system must maintain tolerance to commensal bacteria, foods and self antigens and induce specific response to pathogens. Peripheral tolerance is probably maintained by “tolerogenic” DCs (16, 23). However, how DCs master the development of tolerance and how they are implicated in various human inflammatory and autoimmune diseases remains unclear (1, 2).

Therefore, there is a strong need for a method that generates tolerogenic dendritic cells that can efficiently induce antigen-specific immune tolerance for use in the treatment or the prevention of auto-immune diseases and graft rejection.

### SUMMARY OF THE INVENTION

The present invention relates to a culture medium suitable for inducing dendritic cell differentiation comprising an effective amount of secretory immunoglobulins A (SIgA).

The invention also relates to a method for obtaining a population of tolerogenic dendritic cells wherein said method comprises a step of culturing monocytes with the culture medium of the invention.

The present invention also relates to a population of tolerogenic dendritic cells obtainable by a method of the invention.

The present invention still relates a population of tolerogenic dendritic cells of the invention for use in the treatment or the prevention of an autoimmune or inflammatory disease and also for use in the induction of transplant tolerance.

The present invention further relates to the use of SIgA for the differentiation of monocytes into tolerogenic dendritic cells.

### DETAILED DESCRIPTION OF THE INVENTION

The inventors have demonstrated that is possible to obtain tolerogenic dendritic cells by culturing monocytes in the presence of an amount of secretory immunoglobulins A (SIgA) during their dendritic cell differentiation. They have shown that SIgA interaction with DCs interferes in the molecular programming of these cells. SIgA-primed DCs (DC-SIgA) are unable to undergo Toll-like receptor (TLR)-dependent maturation and become tolerogenic.

### DEFINITIONS

Throughout the specification, several terms are employed and are defined in the following paragraphs.

A “monocyte” is a large mononuclear phagocyte of the peripheral blood. Monocytes vary considerably, ranging in size from 10 to 30 μm in diameter. The nucleus to cytoplasm ratio ranges from 2:1 to 1:1. The nucleus is often band shaped (horseshoe), or reniform (kidney-shaped). It may fold over on top of itself, thus showing brainlike convolutions. No nucleoli are visible. The chromatin pattern is fine, and arranged in skein-like strands. The cytoplasm is abundant and appears blue gray with many fine azurophilic granules, giving a ground glass appearance in Giemsa staining. Vacuoles may be present. More preferably, the expression of specific surface antigens is used to determine whether a cell is a monocyte. For instance, monocytes express CD14 and HLA-DR (monocyte markers) and not CD1c (DC marker), CD56 (NK cell marker), CD19 (B cell marker), CD3 (T cell marker), and CD16b and CD66b (neutrophil markers).

A “dendritic cell” (DC) is an antigen presenting cell existing in vivo, in vitro, ex vivo, or in a host or subject, or which can be derived from a hematopoietic stem cell, a hematopoietic progenitor or a monocyte. DCs and their precursors can be isolated from a variety of lymphoid organs, e.g., spleen, lymph nodes, as well as from bone marrow and peripheral blood. The DCs has a characteristic morphology with thin sheets (lamellipodia) extending in multiple directions away from the DC body. DCs express constitutively both MEW class I and class II molecules, which present peptide antigens to CD8+ and CD4+ T cells respectively. In addition, human skin and mucosal DCs also express the CD1 gene family, MEW class 1-related molecules that present microbial lipid or glycolipid antigens. The DC membrane is also rich in molecules that allow adhesion of T cells (e.g. intercellular adhesion molecule 1 or CD54) or that co-stimulate T-cell activation such as B7-1 and B7-2 (also known as CD80 and CD86 respectively).

An “immature dendritic cell” or “IDC” refers to a cell in a state of differentiation, (from for example monocytes) that has been treated in a specific manner, typically with GM-CSF and IL-4. Immature dendritic cells (or undifferentiated dendritic cells) are characterised by high endocytic activity and low T-cell activation potential. Immature dendritic cells typically show low levels of surface receptors HLA-DR, CD40, CD80, CD83, CD86, DC-Lamp and CCR7. Immature dendritic cells furthermore show high levels of surface receptor CD1a, DC-SIGN, CCR6 and very low levels of the monocyte marker CD14.

As used herein, the term “tolerogenic dendritic cell” refers to a dendritic cell that is derived from a monocyte exposed to a differentiation stimulus, whereby the dendritic cell acquires the ability of inducing tolerance (i.e. capable of silencing or down-modulate an immunological response). Therefore, a tolerogenic dendritic cell has low ability to activate effector T cells but high ability to induce and activate regulatory T cells. Thus, tolerogenic dendritic cells produce high amounts of IL-10 after stimulation with LPS, poly I:C or CTB comparatively to IDCs. Moreover, tolerogenic dendritic cells trigger induction and or expansion of CD4+ CD25+ Foxp3+ regulatory cells (also known as Treg cells). Such regulatory cells play key roles in the maintenance of immunologic self-tolerance and negative control of a variety of physiological and pathological immune responses.

The terms “secretory immunoglobulins A” or “SIgA” are used interchangeably and refer to a biological compound comprising two immunoglobulin A molecules, which are joined by a J-protein, also known as J-chain (joining chain), and secretory component. The J-chain is a polypeptide of molecular mass 15 kD, rich with cysteine and structurally completely different from other immunoglobulin chains. The secretory component is synthesized by epithelial cells of the mucous membrane of gastrointestinal, respiratory and urogenitaltract. (SIgA) is the major immunoglobulin in saliva, tears, colostrum, nasal mucous, mother's milk, tracheobronchial and gastrointestinal secretes and is essential in protecting mucosal surfaces. The contribution of SIgA in the defense of mucosal epithelia plays an important role in preventing pathogen adhesion to host cells, therefore blocking dissemination and further infection. Immune exclusion mechanism represents the dominant mode of action of the SIgA antibody. Furthermore, SIgA have properties extending from intracellular and serosal neutralization of antigens, activation of non-inflammatory pathways and homeostatic control of the endogenous microbiota. Newborns are provided with SIgA by mother's milk and are passively immunized against gastrointestinal infections. SIgA can be purified for example from women breast milk as described in the Examples below.

### Culture Medium of the Invention

In a first aspect of the invention, the present invention relates to a culture medium suitable for inducing dendritic cell differentiation comprising an effective amount of secretory immunoglobulins A (SIgA).

As used herein, the term “culture medium” refers to a liquid medium suitable for the in vitro culture of mammalian cells, in particular human cells. Typically, the culture medium of the invention contains a source of carbon as energy substrate, such as glucose, galactose or sodium pyruvate; essential amino-acids; vitamins, such as biotin, folic acid, B12; inorganic salts; an antioxidant, such as glutathione reduced (GSH), ascorbic acid, etc. The culture medium of the invention may be based on a commercially available medium such as RPMI1640 from Invitrogen. Preferred media formulations that will support the growth and the differentiation of monocytes into DCs include chemically defined medium (CDM).

As used herein, the term “chemically defined medium” refers to a nutritive solution for culturing cells, in particular monocytes, which contains only specified components, preferably components of known chemical structure. A chemically defined medium is free of animal-derived substances. In a particular embodiment, the culture medium of the invention consists essentially of synthetic compounds, compounds of human origin and water. Advantageously, said culture medium can be used for culturing cells according to good manufacturing practices (under “GMP” conditions).

As used herein, the term “culture medium suitable for inducing dendritic cell differentiation” refers to any medium capable of supporting the dendritic cell differentiation of cells, in particular monocytes, and the growth of the obtained dendritic cells.

Said culture medium supports the growth and the differentiation of monocytes into dendritic cells (DCs). Monocytes may be differentiated into DCs by any technique well known in the art. In a particular embodiment, granulocyte-macrophage colony-stimulating factor (GM-CSF) and interleukin-4 (IL-4) are added to a culture medium in order to differentiate monocytes into DCs.

It should be further noted that dendritic cells (DCs) may also be derived from a hematopoietic stem cell or a hematopoietic progenitor.

An effective amount of secretory immunoglobulins A (SIgA) is added to the culture medium suitable for dendritic cell differentiation.

In one embodiment, SIgA is added in a concentration ranging from 1 to 500 μg/ml, preferably ranging from 10 to 250 μg/ml and more preferably at about 100 μg/ml.

In another embodiment, SIgA mammal SIgA, in particular human SIgA.

### Methods for Obtaining a Population of Tolerogenic Dendritic Cells

In a second aspect, the present invention provides a method for obtaining a population of tolerogenic dendritic cells wherein said method comprises a step of culturing monocytes with the culture medium of the invention.

The monocytes that serve as starting material may be isolated according to any technique known in the art. For instance, monocytes were isolated by negative selection using magnetic immunobeads (Monocyte Isolation Kit II, Miltenyi). The purity of sorted monocytes was >98%, as indicating by the expression of CD14 and HLA-DR (monocyte markers) and absence of CD1a and CD1c (DC marker), CD56 (NK cell marker), CD19 (B cell marker), CD3 (T cell marker), and CD16b and CD66b (neutrophil markers) expression.

The step of culturing monocytes with the culture medium of the invention shall be carried out for the necessary time required for the dendritic differentiation of monocytes until the obtention of tolerogenic dendritic cells. Typically, the culture of monocytes with a culture medium of the invention shall be carried out for at least 2 days, preferably at least 4 days, even more preferably at least 6 days.

The culture medium of the invention has to be renewed, partly or totally, at regular intervals. Typically, the culture medium of the invention can be replaced with fresh culture medium every other day, for 6 days.

The invention also relates to the use of SIgA for the differentiation of monocytes into tolerogenic dendritic cells.

### Populations of Tolerogenic Dendritic Cells Obtained According to a Method of the Invention and Pharmaceutical Compositions Thereof

In another aspect, the present invention also relates to a population of tolerogenic dendritic cells obtainable by a method as defined above.

Advantageously, said population of tolerogenic dendritic cells is homogenous, i.e. it is not necessary to perform any sorting or selection to isolate the tolerogenic dendritic cells-from other contaminating cells.

Typically, the population of tolerogenic dendritic cells according to the invention has a purity of at least 95%, preferably 99%, even more preferably 100%.

In one embodiment, the population of tolerogenic dendritic cells may be pulsed with an antigen of interest. To achieve antigen presentation by the tolerogenic dendritic cells, the antigen of interest is provided in an amount effective to “prime” the tolerogenic dendritic cells to express antigen-pulsed MHC class I and/or class II antigens on the cell surface (in order to obtain antigen-pulsed tolerogenic dendritic cells).

As used herein, the term “pulsed” refers to the process by which the tolerogenic dendritic cells may be “loaded” with an antigen of interest (i.e. MOG35-55 peptide).

The present invention also provides a pharmaceutical composition comprising the population of tolerogenic dendritic cells according to the invention. The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like. This pharmaceutical composition can contain additional additives such as mannitol, dextran, sugar, glycine, lactose or polyvinylpyrrolidone or other additives such as antioxidants or inert gas, stabilizers or recombinant proteins (e.g. human serum albumin) suitable for in vivo administration.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

### Therapeutic Methods and Uses

Another aspect of the invention relates to a population of tolerogenic dendritic cells or a pharmaceutical composition of the invention for use in the treatment or the prevention of an autoimmune or inflammatory disease.

The invention also relates to a method for treating an autoimmune or inflammatory disease comprising the step of administering a pharmaceutically effective amount of a population of tolerogenic dendritic cells of the invention to a patient in need thereof.

In one embodiment, the autoimmune or inflammatory disease is selected from the group consisting of allergy, multiple sclerosis, auto-immune type 1 diabetes (diabetes mellitus), rheumatoid arthritis, inflammatory bowel disease, systemic lupus erythematosus, Graves' disease and nephropathy, vasculitides, scleroderma, psoriasis, autoimmune thyroide disease, glomerulonephritis and Sjogren's disease.

In another embodiment, the population of tolerogenic dendritic cells may be pulsed with an antigen of interest as described above.

Another aspect of the invention relates to a population of tolerogenic dendritic cells or a pharmaceutical composition of the invention for use in the induction of transplant tolerance.

The invention also relates to a method for induction of transplant tolerance comprising the step of administering a pharmaceutically effective amount of a population of tolerogenic dendritic cells of the invention to a patient in need thereof.

In the context of the invention, the terms “treating” or “treatment”, as used herein, refer to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “pharmaceutically effective amount” refers to any amount of a population of tolerogenic dendritic cells according to the invention (or a pharmaceutical composition thereof) that is sufficient to achieve the intended purpose.

As used herein, the term “subject” denotes a mammal, such as a rodent, a feline, a canine, and a primate. Preferably a subject according to the invention is a human.

Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

For therapy, tolerogenic dendritic cells and pharmaceutical compositions according to the invention may be administered through different routes. The dose and the number of administrations can be optimized by those skilled in the art in a known manner.

The invention will be further illustrated by the following figures and examples. However, these examples and figures should not be interpreted in any way as limiting the scope of the present invention.

### FIGURES

FIG. 1: Phenotypic analysis of differentiation of Human monocytes to iDC in the presence or not SIgA. At day 6, cells were collected in PBS/azidothymidine 0.01%/BSA 0.5% and incubated with mAb directed against CD83, CD1a, CD40, CD86 and CD83. Data are expressed as the mean percentage (%) of positive cells and MFI (Mean Fluorescence Intensity) for one representative outcome of five separate experiments. percentage of positive cells is indicated in corresponding quadrant.

FIG. 2: Effect of SIgA treatment on the maturation and migration markers of DCs stimulated with LPS. At day 6, DC differenciated in the presence or not of SIgA were stimulated with LPS for 48 h before to be washed and collected in PBS/azidothymidine 0.01%/BSA 0.5%. Cells were then stained with mAb directed against CD83, CD1a, CD40, CD86, CD83. Data are expressed as the mean percentage of positive cells (%) and MFI (Mean Fluorescence Intensity) for one representative outcome of five separate experiments.

FIG. 3: Effect of SIgA treatment on the production of cytokines by DCs stimulated with LPS, poly I:C and the B subunit cholera toxin (CTB). Cytokines were quantified in cell-free culture supernatants of DCs stimulated with LPS (1 μg/ml) or CTB (1 μg/ml) for 48 h, using either sandwich immunoassay kits purchased from eBioscience (France) or BD™ Cytometric Bead Array (BD Biosciences). IL-10, IL-12, IL-6 and IL-23. Data represents the means and SD of three independent experiments (**p<0.001 and *p<0.05).

FIG. 4: Effect of SIgA treatment on phagocytosis capacity of Human dendritic cells. At day 6 of differentiation, SIgA treated and untreated DCs (5×105 cells) were incubated with a suspension of phycoerythrin (PE) 1 μm diameter latex sulfate beads (Molecular probes) for 2 h at a 0.05% dilution. Dendritic cells were then washed and analyzed on a FACS Calibur™ and the CellQUEST software. The result is expressed as the mean±SD of Mean Intensity of Fluorescence (MFI) obtained for three experiments. (*p<0.05).

FIG. 5: Effect of SIgA on Human dendritic cells survival. At day 6 of differentiation, SIgA-treated and untreated immature dendritic cells were further cultured in the presence or not of additional doses of SIgA. DCs viability was determined by a dye exclusion method using trypan blue to stain dead cells. Results were expressed as percent ±SD of survival cells obtained for three experiments.

FIG. 6: Effect of treatment of DC with SIgA on the capability to induce an extension/generation of CD4/CD25 T cells expressing Foxp3: SIgA treated or untreated immature dendritic cells were incubated with LPS (1 μg/ml) for 2 hours at 37° C. Foxp3 expression in the T cells cultured with DC treated or not with SIgA. At day 6 of DC-T coculture, total RNA was extracted, before performing reverse transcription and real time PCR assays on Foxp3 gene as depicted in the method section. Results are expressed in arbitrary units ±SD of the ratios between the target Foxp3 and the GAPDH housekeeping mRNA. Data presented were obtained for three experiments.

FIG. 7: Effect of SIgA treatment on the maturation of mice DC stimulated with LPS. At day 8, DC differenciated from cell suspensions prepared from spleen in the presence or not of SIgA were stimulated with LPS for 48 h before to be washed and collected in PBS/azidothymidine 0.01%/BSA 0.5%. Cells were then stained for CD83, CD86, CD80, CD40, MHC classe I and classe II. Data are expressed as the means percentages of positif cells ±SD obtained for three experiment. (*p<0.05).

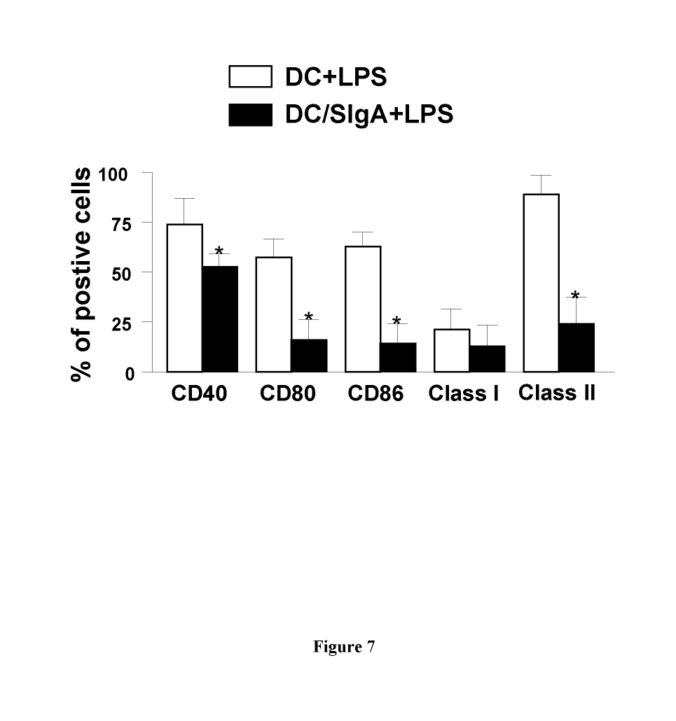

FIG. 8: Effect of SIgA treatment of mice DC on production of IL-10 and induction of regulatory T cells (TGF-β+ and Foxp3+). At day 8, DC differenciated in the presence or not of SIgA were stimulated with LPS for 48 h and IL-10 estimated. The induction of CD4+ CD25+FOXP3+ regulatory T cells expansion/proliferation has been used to evaluate the tolerogenic potential of DCs. SIgA-DC were co-cultured with naïve monoclonal T cells specific for ovalbumine peptide (DT11) (B) or specific for a pancreatic beta cell antigen (BDC) (C) in the presence or in the absence of TGF-β. Cells were then stained for Foxp3 and TCR. Data are expressed as the means percentages of positif cells ±SD obtained for three experiment. (*p<0.05).

FIG. 9: Therapeutic potential of SIgA treated DCs in experimental auto-immune encephalomyelitis. DCs or SIgA-DCs were loaded with MOG35-55 peptide and injected 7 days before MOG35-55 immunization (MOG35-55-immunized NOD mice develop an experimental auto-immune encephalomyelitis (EAE) drove by an I-Ag7-restricted CD4+ T cells. Three groups of 15 mices each were injected either with medium, DC or SIgA treated DC. Clinical symptoms were monitored daily after immunization. The clinical score was graded as follows: 0, no disease; 1, tail limpness; 2, hind limb weakness; 3, hind limb paralysis; 4, fore limb weakness; 5, quadriplegia; 6, death. Cumulative disease scores were calculated by adding daily disease scores from the day after immunization until the end of the experiment.

FIG. 10: Therapeutic potential of SIgA treated DCs in auto-immune type 1 diabetes. We validated the therapeutic potential of SIgA-DCs to prevent diabetes induced by the transfer of diabetogenic T cells (BDC) in T cell deficient mice (NOD CaKo). Three groups of ten mices each, were injected of either DBC alone, DBC/DCs or DBC/SIgA-DCs co-cultures. For diabetes diagnosis, mice were tested daily from day 5 from disease onset, using Glukotest and Haemoglukotest kits (Boehringer, Mannheim, Germany). Diabetes is induced 10 days after diabetogenic T cell transfer (A). In this model, induction of regulatory T cells (Foxp3+) by DCs treated or not with SIgA was analysed in pancreatic (PLN), mesenteric lymph nodes (MLN) and spleen (B). Intracellular staining for IFN-γ and IL-10 in cells infiltrating PLN was carried out as described in the method section (C). Data are expressed as percentage of positive cells for one representative outcome of three separate experiments.

### EXAMPLE

Material & Methods

Antibodies and Reagents:

Recombinant human interleukine-4 (IL-4), IL-2, granulocyte macrophage-colony stimulating factor (GM-CSF) were obtained from Tebu (Tebu; Santa Cruz Biotechnology, California, US), Fluorescein Isothyocyanate (FITC)-conjugated anti-CD40, CCR7, CD80, Ig-alpha chain, CD86, CD4 and CD25 and PE-anti-CCR 6 were obtained from Becton Dickinson (Le Pont de Claix, France). The FITC-conjugated anti-CD83, IgG1 and the PE-conjugated anti-CD1a and IgG1 were from Immunotech (Marseille, France). The polyclonal anti-human heavy alpha chain, Lipopolysacharide (LPS) (Escherichia coli), Cholera toxin B subunit and Phytohemaglutinin A (PHA) were purchased from Sigma (St Quentin Fallavier, France). Poly(I:C) was from R & D Systems, Oxford, UK. Phycoerythrin (PE) 1 μm diameter latex sulfate beads were obtained from Molecular probes (France). Healthy women breast milk samples were collected at the lactarium of the Institut de Puériculture (Paris, France).

Purification of SIgA from Women Breast Milk:

The anti-human heavy alpha chain was coupled to activated Sepharose 4B according to the manufacturer's instructions (Pharmacia Biotech). Pools (100 ml) of breast milk samples from healthy women were allowed to interact with the matrix overnight at 4° C. before extensive washing of the column with PBS until the optical density of the effluent reached 0.001. The column was then eluted with glycine-HCl 0.2 M, pH 2.5. The pH of eluted material was rapidly neutralized with 1 M Tris-HCl, pH 8.3 and further dialyzed against PBS overnight. To quantify the purified IgA, plastic plates were coated with the anti-alpha chain (3 μg/ml) in PBS, at pH 7.4 overnight at 4° C. The plates were washed with PBS-Tween 0.1% prior to saturation with PBS-skim milk 2%. Dilutions of breast milk purified IgA were then added and incubated 1 hour at 37° C. After washing, peroxidase-labeled goat anti-alpha and goat anti-secretory composante (SC) antibodies were added for 1 h at 37° C. before addition of substrate. For standard curve, the same experiment was done using breast-milk SIgA previously quantified. All preparations were controlled by ELISA and SDS-PAGE.

Human Monocytes Derived Dendritic Cells Preparation and SIgA Treatment procedure:

DCs were differentiated from PBMC. After step-density gradient centrifugation, PBMC (10⁷ cells/ml) were cultured in RPMI 1640/10% of normal human serum for 1 h at 37° C. After several washing, adherent cells were maintained in RPMI 1640/10% FCS, 1% antibiotics supplemented with IL-4/GM-CSF (both at 10 ng/ml) to obtain iDC. Medium was changed every 48 h and new cytokines IL-4/GM-CSF added to the medium. Contamination of iDCs with CD3⁺ T lymphocytes was <1% as checked by FACS. Autologous lymphocytes represented by the nonadherent cell fraction of PBMC were washed before to be freezed. To investigate the effect of SIgA on differentiation of dendritic cells, purified SIgA used at 100 μg/ml were added every 48 h to IL4/GM-CSF-monocytes cultures. For negative control, cells were incubated in RPMI/10% FCS 1% antibiotics in the absence of any additional immunoglobulins.

Preparation of Mice Primary DCs:

Bone marrow-derived DCs were prepared from the femurs of male or female mice between 6 and 8 wk of age. DC precursors were plated on six-well lowcluster plates in RPMI 1640 medium containing 10% FBS and penicillin/streptomycin (base medium), and 10 ng/ml murine GM-CSF (R&D). On the fourth day of culture, culture medium and SigA (100 μg/mL) were added on days 4-5 and. In some conditions LPS (1 μg/ml) was added on day 7. For all experiments DC were harvested on day 8 of the cultures.

Stimulation of Human Dendritic Cells with LPS, Poly I:C and CTB:

Immature dendritic cells (10⁶ cells) obtained at 6 days of culture cells in the presence of SIgA or Igs free medium as control, were incubated in the absence, or in the presence of 1 μg/ml of lipopolysaccharide (LPS), polyinosinic:polycytidylic acid (poly I:C) or cholera toxin B (CTB) for additional 48 h or 24 h at 37° C.

Human Dendritic Cells Immunostaining and Flow Cytometry Analysis:

The expression of maturation markers by dendritic cells was assessed by cytofluorometry using a FACS Calibur™ and the CellQUEST Software™ (Becton Dickinson). Briefly, cells (1×10⁶ cells per test) were incubated with directly conjugated mAbs against membrane molecules or with matched isotypes for 30 min at 4° C., washed with (PBS/NaN3 0.05%) and fixed with 1% of paraformaldehyde before analysis. In some experiments, cells were incubated for an additional step during 30 min with conjugated-secondary antibodies before fixation step. For intracellular staining, cells were fixed with 4% of paraformaldehyde for 15 min at 4° C., washed twice in PBS/NaN3 0.05%/BSA 0.2% supplemented with saponin (0.5%) for cells permeabilization and then incubated with antibodies for 30 min at room temperature. After washes, cells were analyzed by FACS Calibur™

Analysis of Mice Dendritic Cells Phenotype by Flow Cytometry:

Cell suspensions were prepared from spleen, PLN and pancreas. Cells were stained at 4° C. in PBS containing 2% FCS and 1% EDTA after blocking FcγR with 2.4G2 mAb. Surface staining was performed with antibodies all from BD Pharmingen. 120G8 mAb was FITC-conjugated in the laboratory. For CD1d tetramer preparation, biotinylated soluble CD1d was loaded with α-GalCer, then incubated with allophycocyanin-conjugated streptavidin. H2D^{b}-NP₃₉₆₋₄₀₄ dextramers were purchased from DakoCytomation. To detect degranulation, anti-CD107a mAb (1D4B; Pharmingen) was added during re-stimulation with NP₃₉₆₋₄₀₄ peptide. For IFN-γ (XMG1.2; BD Pharmingen) intracellular staining, single-cell suspensions were stimulated with 1 μg/ml of viral NP₃₉₆₋₄₀₄ peptide for 5 h at 37° C. in the presence of 10 U/ml recombinant mouse IL-2 (R&D) and 1 μg/ml brefeldin A. For IL-10 and TGF-β (BD Pharmingen) intracellular staining of CD4⁺ T cells, single-cell suspensions were incubated with PMA, ionomycine and Brefeldine A (all from Sigma) for 5 h at 37° C. Treg cells were detected using the anti-mouse/rat Foxp3 staining set (FJK-16s, eBioscience). Stained cells were analyzed on a FACSCalibur flow cytometer (BD Biosciences).

Effect of SIgA Treatment on Phagocytosis Capacity of Human Dendritic Cells:

To analyze the effect of SIgA treatment during dendritic cells differentiation on the phagocytosis capacity of immature dendritic cells, SIgA treated and untreated cells (5×10⁵ cells) were incubated with a suspension of phycoerythrin (PE) 1 μm diameter latex sulfate beads (Molecular probes) for 2 h at a 0.05% dilution. Dendritic cells were then washed three times in PBS/NaN3 0.05% and analyzed on a FACS Calibur™ and the CellQUEST software. The result is expressed as the mean±SD of Mean Intensity of Fluorescence (MFI) obtained for three experiments.

Effect of SIgA on Cytokines Release by Human Dendritic Cells:

The cytokines IL-10, IL-6, IL-12 and IL-23 were quantified in cell-free culture supernatants using sandwich immunoassay kits purchased from eBioscience (France). The Human IL-12 p70 ELISA reagent set used, specifically measures the bioactive, heterodimeric form of IL-12, p70, without interference by p40 monomer, homodimer, or IL-23 (p19/p40). The Human IL-23 ELISA uses a p19-specific capture antibody and a p40-specific detection antibody that renders this sandwich ELISA exclusively specific for Human IL-23. Human TNFα, IL-1b, IL-8, RANTES, MIG and MCP1 were quantified by mean of BD™ Cytometric Bead Array (CBA) using the Human Inflammatory Cytokine Kit (551811) and Human Chemokines Kit (552990) (BD Biosciences).

Effect of SIgA on Human Dendritic Cells Survival:

SIgA-treated or untreated immature dendritic cells at differentiation step were further cultured in the complete RPMI 1640 medium supplemented with 10% FCS in the presence or not of additional doses of SIgA every 3 days during 3 weeks. DCs viability was determined by a dye exclusion method using trypan blue to stain dead cells. Results of three experiments were expressed as percent of Survival Cells.

Microscopy Staining of SIgA in Human DCs:

The binding of secretory IgA to immature dendritic cells was also determined with fluorescence microscopy. Briefly, SIgA-treated or untreated immature dendritic cells (1×10⁵) were incubated with anti-alpha antibodies for 30 min at 4° C. prior to be washed and fixed with 1% paraformaldehyde (PFA). After several washes, a drop of cells was then adsorbed on microscopy adapted slide for 5-15 min at room temperature. The coverslids were mounted in Mowiol (Sigma, St. Louis, Mo.) and observed by confocal microscopy using a Leica microscope (Leica, Wetzlar, Germany).

Effect of SIgA Treatment on the Capability of Dendritic Cells to Induce Extension/Generation of CD4/CD25 T Cells and Foxp3 Expression:

The SIgA treated or not dendritic cells were examined for their ability to amplify allogenic CD4/CD25 lymphocytes population. SIgA treated or untreated immature dendritic cells were incubated with LPS (1 μg/ml) for 2 hours at 37° C. before several washing and addition of autologous unstimulated T cells. After 6 days of co-culture, cells were analysed by FACS in order to determine the percent of CD4+ CD25+ T cells.

Total RNA was extracted at day 6 from a coculture of SIgA-treated or not DC with autologous T cells using the RNeasy Mini Kit, according to manufacturer's instructions (Qiagen AG, Basel, Switzerland), and purity was determined by spectrophotometry. For each sample, 1 μg of total RNA was reverse transcribed into first-strand cDNA in a 20-μL final volume containing 1 μM random hexanucleotide primers, 1 μM oligo dT, 200 U Molony murine leukemia virus reverse transcriptase (Promega).

Real time PCR assays were performed on the LightCycler apparatus (Roche Diagnostics, Meylan, France), using primers designed with Primer3 software and selected to differentiate between amplification of cDNA and contaminating genomic DNA. PCR reactions were performed in 20 μl, using the Faststart DNA Master SYBR Green I kit (final MgCl2 concentration, 2.5 mM) (Roche Diagnostics) according the following thermal condition: 95° C. for 10 min; 45 cycles of 95° C. for 15 s, 62° C. for 20 s, 72° C. for 20 s, followed by melting curve analysis. All reactions were performed in triplicate. Data analysis was performed with the LightCycler 1.0 software. The threshold level was determined by the software according to the optimization of the baseline and the standard curve. Standards were obtained by amplification of a control sample in a PCR reaction, using the same primers, reagents and conditions optimized for the real time analysis. Arbitrary quantity values were assigned to the resulting standard and 4-fold serial dilution were made to obtain a 8-points standard curve. Results are presented as ratios between the target gene mRNA and the GAPDH housekeeping mRNA.

Determination of the Role of CD89 and CD71 in the Induction of Tolerogenic Dendritic Cells by SIgA:

Human monocytes were differentiated in the presence of SIgA in the presence or not of human recombinant CD89 or recombinant CD71 both at 50 μg/ml, in the presence of GM-CSF and IL-4. At day 6 of differentiation, cells were stimulated with LPS for 48 hours before to be washed three times in PBS/NaN3 0.05%, stained for CD83 and CD80 expression and analyzed on a FACS Calibur™ and the CellQUEST software.

Effect of SIgA Treatment on the Activation of NFkB Transactivator Factor in DC Stimulated with LPS:

DNA Pull Down. 10⁷ untreated or SIgA treated DCs were stimulated with LPS (1 μg/ml) for 2 hours. The cytosolic fraction was extracted thanks buffer A (10 mM HEPES-NaOH pH 7.6, 3 mM MgCl₂, 10 mM KCl, 5% glycerol, 0.1% Nonidet P-40, 1 mM Vanadate, 10 mM NaF, 1 mM Sodium pyrophosphate and 25 mM β-glycerophosphate) containing Complete Proteinase Inhibitors Cocktail Tablets 25X (Roche). Cells were incubated 10 min at +4° C. and centrifuged at 10,000 rpm for 2 min. Nuclear proteins were obtained by traitment on the pellet by buffer B (buffer A, 1 mM Vanadate, 10 mM NaF, 1 mM Sodium pyrophosphate 25 mM β-glycerophosphate and 300 mM KCl) and centrifuged after 30 min at +4° C. at 15,000 rpm for 20 min. Finally, nuclear fraction was diluted in v/v buffer A. Nuclear extract was incubated with 1 μg of double strand probe biotyniled in 5′ for 1 hour at +4° C. 25 μl of sepharose beads (Streptavidin Hight Performance GE Healthcare) were used to catch biotyniled probe (15 min at +4° C.). Protein associated with the probes were eluated at 95° C. for 5 min in Laemmli Buffer (62.5 mM Tris-HCl pH 6.8, 2% SDS, 10% glycerol, 1 mM sodium orthovanadate, 5% DTT and Bromophenol blue). Eluated proteins were loaded into 10% denatured polyacrylamide gels with 1X TG-SDS 1%. Proteins were transferred to nitrocellulose membrane after subjection to SDS-PAGE. The membrane was incubated in blocking buffer (1×TBS and 0.1% Tween 20 with 5% nonfat dry milk) for 1 h at room temperature. Incubation overnight at 4° C. with primary Abs anti-p50 (Stressgen), anti-c-Rel (Santa Cruz Biotechnology) and anti-p65 (Cell Signaling) was followed by incubation with HRP-conjugated secondary Ab for 1 h at room temperature. Proteins were detected by adding (ECL Plus Western Blotting Detection reagents, Amersham) and exposure to x-ray film (CL-XPosure™ Film, PIERCE).

Diabetes Diagnosis and EAE Induction and Clinical Evaluation:

In Experimental Autoimmune Encephalomyelitis (EAE) experiments, BMDCs were incubated with 100 μg/ml MOG35-55 peptide in complete medium for 4 h at 37° C. before disease induction. After intensive washing, MOG35-55 peptide-pulsed DCs (5×10⁵) were injected i.v. into NOD mice on day −7 before EAE induction (day 0). Mice were then injected s.c. with 200 μg of MOG35-55 peptide in 100 μL of PBS emulsified with 100 μL of CFA and further enriched with 5 mg/ml M. tuberculosis (H37Ra). In addition, 500 ng of pertussis toxin was injected i.p. on day 0 and day 2. Clinical symptoms were monitored daily after immunization. The clinical score was graded as follows: 0, no disease; 1, tail limpness; 2, hind limb weakness; 3, hind limb paralysis; 4, fore limb weakness; 5, quadriplegia; 6, death. Cumulative disease scores were calculated by adding daily disease scores from the day after immunization until the end of the experiment. For diabetes diagnosis, mice were tested daily from day 5 from disease onset, using Glukotest and Haemoglukotest kits (Boehringer, Mannheim, Germany).

Isolation and Transfer of Mouse BDC2.5 Cells and In Vitro Stimulation:

BDC2.5 cells were obtained from BDC2.5 Cα−/− NOD or Thy1.1 BDC2.5 Cα−/− NOD mice before they developed diabetes (5-7 weeks of age). Splenocyte suspensions were prepared, and red cells and B cells were removed by hypotonic lysis and by sheep anti-mouse IgG beads (Dynal, Oslo, Norway). CD62L+ splenocytes were positively selected with biotinylated anti-CD62L mAb and Streptavidin (SA) microbeads (Miltenyi Biotec, Auburn, Calif.). CD4+ T cells were transferred, 1.5×10⁵ (doses of BDC2.5 T cells consistently induced diabetes in Cα^{-/-} recipient). All recipient mice were used for BDC2.5 T cell transfer at 6-7 weeks of age. For in vitro stimulation, CD62L+ BDC2.5 cells (5×104) were incubated with in the presence of BMDCs (5×10⁴) and 50 units/ml of recombinant human IL-2. In some experiments 1 ng/ml of recombinant TGF-β was added.

Statistical Analysis:

Data are represented as mean±SEM of n determination. Standard t test or nonparametric Mann-Whitney were used to determine the statistical significance of the data at the individual time points. When analyzing experiments performed at multiple time points, ANOVA for repeated measurement statistics were used to calculate the overall significance and average difference between the two groups. A P value less than 0.05 was considered significant.

Results

Dendritic Cells Differentiated from Monocytes in the Presence of SIgA have an iDC Phenotype:

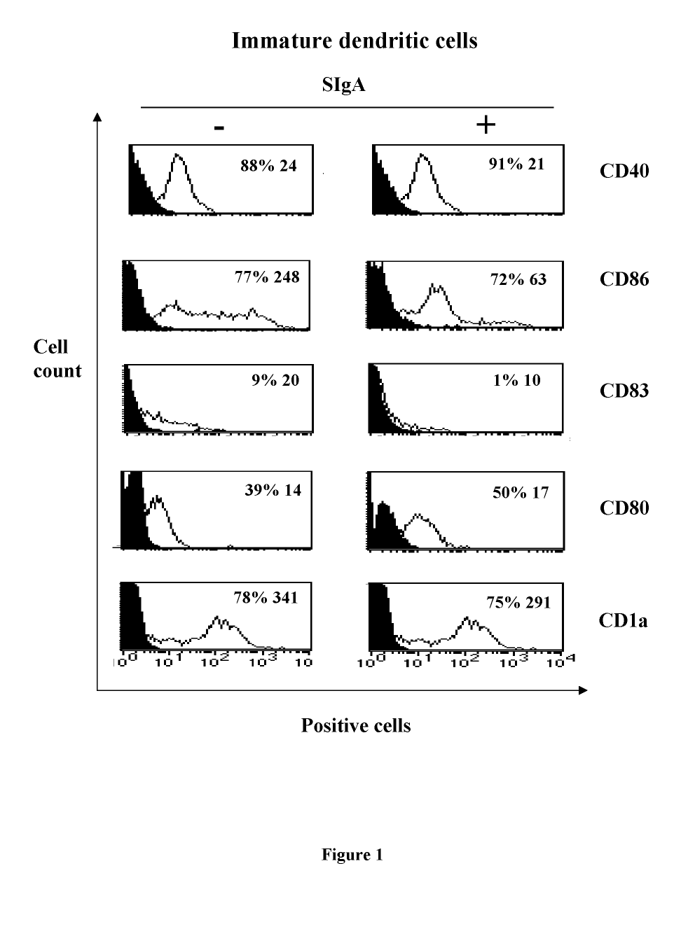

Treatment of human monocytes with SIgA induces differentiation into cells with a phenotype of immature dendritic cells. Cells obtained after 7 days of differentiation of monocytes in the presence of GM-CSF, IL-4 and SIgA express CD1a, CD80, CD40, CD86 and CD83low (FIG. 1). Morphological observation showed that SIgA treated immature dendritic cells formed cellular clusters similar to those observed with untreated cells. Immunofluorescence staining using anti-IgA-FITC antibodies showed that DCs bind and internalize SIgA molecules.

Treatment of Monocytes with SIgA Inhibits Induction by LPS of Maturation of Dendritic Cells:

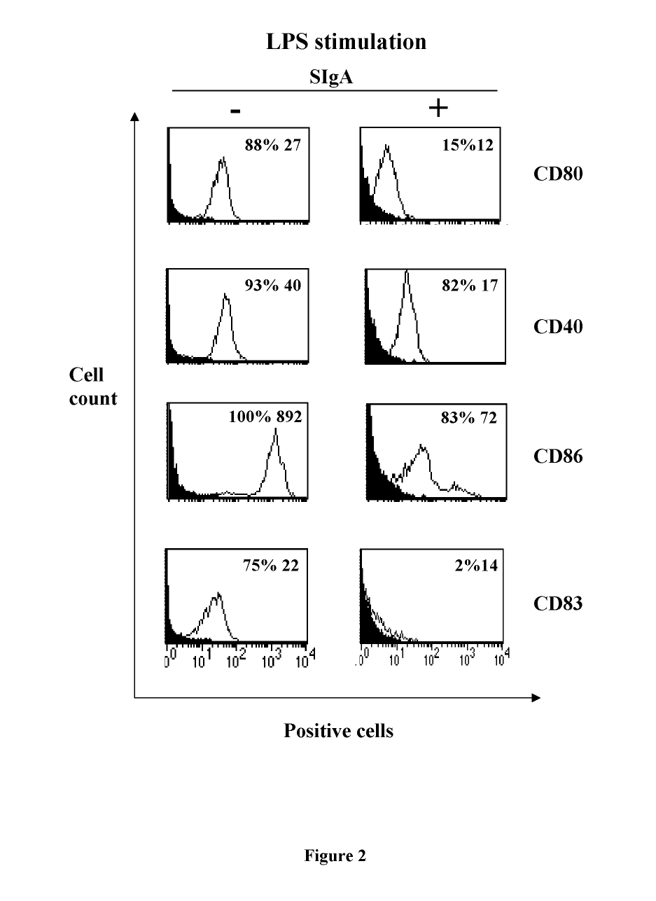

Human dendritic cells obtained at day 7 of differentiation in the presence or absence of SIgA were stimulated by LPS (1 μg/ml) for 48 hours before being harvested and stained for analysis by flow cytometry. Our results show that cells treated by SIgA do not mature after stimulation by LPS. Indeed, SIgA induce a significant down expression of either the percent or the mean or both of fluorescence intensity (MFI) of maturation and costimulatory markers CD83, CD80, CD86 and CD40 (FIG. 2). Furthermore, we investigated the expression of CCR7 molecule on DCs, which is a marker of both maturation and migration capacity to lymphoide organs of DCs. Our data showed that SIgA induced a down expression of CCR7 (from 85.2% to 17.9%) on DCs stimulated by LPS, compared to SIgA untreated cells. Incontrast, no significant modulation of CCR6 molecules, that is a marker of resident cells, was observed.

Stimulation of SIgA-Treated Dendritic Cells by LPS, PolyI:C and CTB Induces Secretion of IL-10 and Inhibition of IL-12 and IL-23:

Dendritic cells obtained at day 7 of differentiation in the presence or absence of SIgA were stimulated by LPS (1 μg/ml), polyI:C (1 μg/ml) and CTB (1 μg/ml) for 48 h. Culture supernatants were then recovered and the secretion of different cytokines was estimated. Our results show that when cells are not treated by the SIgA during their differentiation into dendritic cells, stimulation by LPS, poly(I:C) or CTB induced significant production of IL-12, IL-23 and IL-6 and very low amount of IL-10. This profile is completely changed when the cells are treated by the SIgA during their differentiation into dendritic cells. Indeed, we observed a significant increase in the production of IL-10, by a factor of 6 for the stimulation with LPS, of 5 for poly(I:C) and 28 with CTB compared to the values obtained with SIgA-DCs. In contrast, the production of IL-12 decreased when the dendritic cells are treated with SIgA, by a factor of 4 when cells were stimulated with LPS and a factor of 2 with CTB. Poly(I:C) stimulation of DCs induce a very low secretion of IL-12 without significant effect of SIgA-treatment.

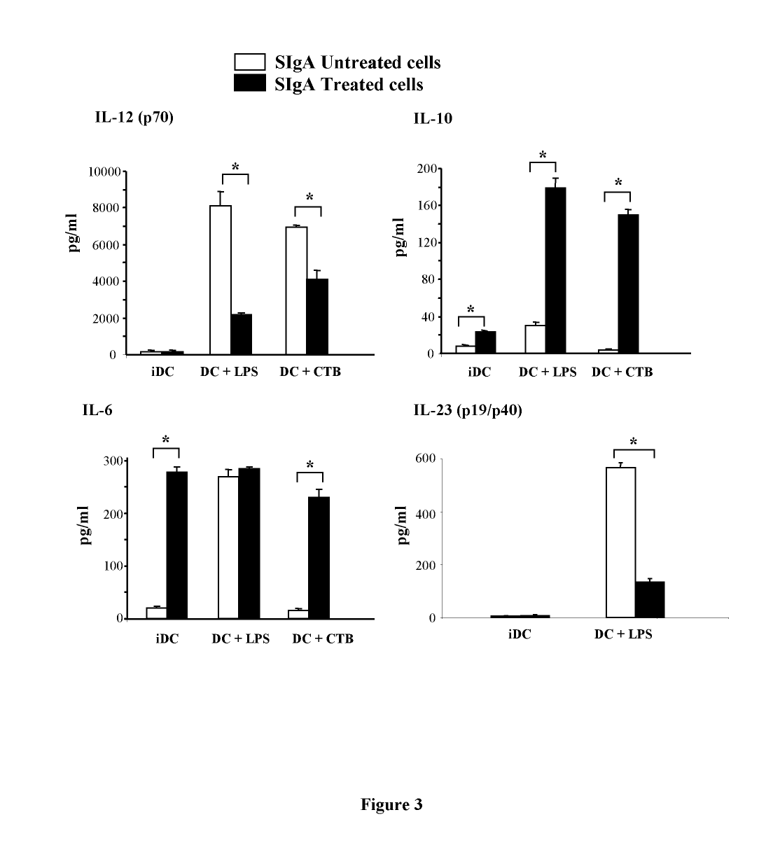

We also observed a decrease of IL-23 (p19/p40) secretion by a factor of 17 when dendritic cells were treated with SIgA before to be stimulated with LPS. A significant increase of 20 fold in the production of IL-6 was observed only when the SIgA treated dendritic cells were stimulated with CTB (FIG. 3).

Stimulation of SIgA-Treated Dendritic Cells by LPS as a Ligand of TLR4 and Poly(I:C) as a Ligand of TLR3 Induced a Differential Effect on Proinflammatory Cytokines:

Stimulation with LPS of DCs differentiated in the presence of SIgA induced a significant decrease of TNFα (from 134 pg/ml to 15 pg/ml). However, no significant difference was observed in TNFα secretion when DCs were stimulated with poly(I:C). In contrast, stimulation with LPS and poly(I:C) of SIgA treated DCs induced a significant increase of IL-1b (by a factor of 8 for LPS and 9 for poly(I:C)).

Stimulation of SIgA-Treated Dendritic Cells by LPS and Poly(I:C) Induced a Differential Effect on Secreted Lymphokines:

Stimulation of SIgA treated DCs with LPS induced a statistically significant decrease of RANTES (from 1287 to 21 ng/ml), of MIG (Monokine induced by gamma interferon) from 65528 to 33 ng/ml and of MCP1 (monocyte chemotactic protein-1) from 1002 to 238 pg/ml). However, no significant difference was observed in IL-8 secretion. In contrast, stimulation of SIgA treated DCs with poly(I:C) induced an increase of IL-8 secretion from 1958 to 25247 pg/ml. As for LPS stimulation, poly(I:C) induced a decrease of MIG secretion from 12456 to 300 pg/ml. No change in the secretion of RANTES and MCP1 by SIgA treated DCs stimulated with poly(I:C).

Treatment of Monocytes with SIgA Induces Immature Dendritic Cells with a High Power of Phagocytosis:

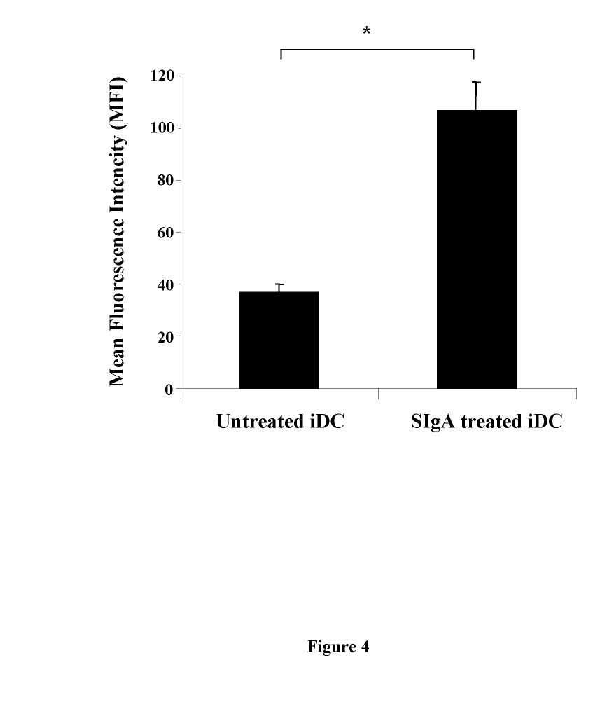

We analyzed the effect of treatment of monocytes with SIgA on the phagocytosis capacity of immature dendritic cells obtained at day 7 of differentiation. Cells (5×105 cells) were incubated with a suspension of latex beads coupled to phytoerythrin (PE) (Molecular Probes, USA) for 2 hours. Cells were then washed and analyzed by flow cytometry. Our results show that treatment of dendritic cells with SIgA induce a high capacity of phagocytosis. Indeed, SIgA treated dendritic cells captured 2 folds higher latex beads coupled to phytoerythrin (PE) compared to untreated cells (106 vs 38 MFI) (FIG. 4).

Treatment with SIgA Induces a Longer Survival Immature Dendritic Cells:

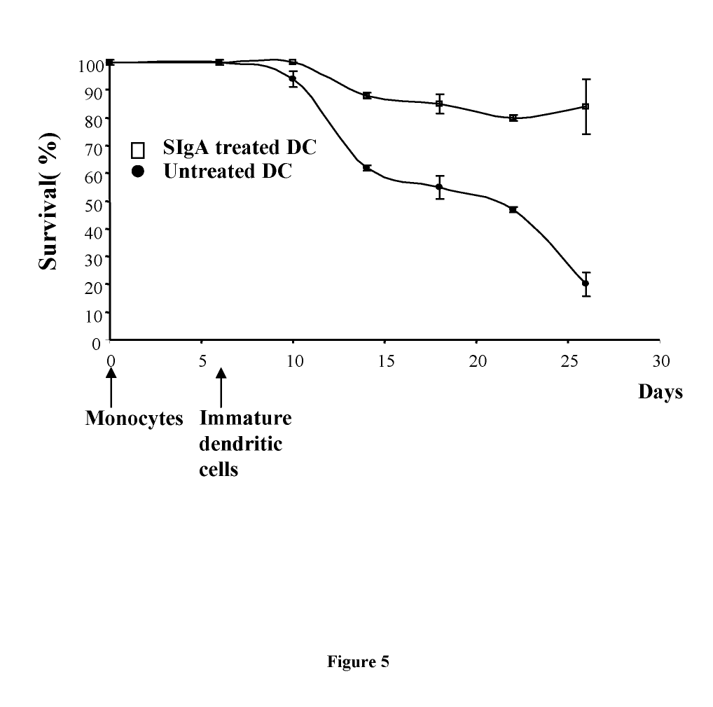

We analyzed the effect of treatment of monocytes by SIgA in the presence of IL-4 and GM-CSF on the survival of immature dendritic cells obtained at day 7 of differentiataion. The viability of dendritic cells was determined by counting cells with trypan blue and the results expressed as percentage of living cells compared to total cells (living+dead cells). Our results show that dendritic cells treated with SIgA have a significant prolonged survival compared to untreated cells. Indeed, at 25 days of culture, approximately 80% of the cells treated with SIgA were alive while this percentage was only of 20% for untreated cells (FIG. 5).

Dendritic Cells Treated with SIgA Promote the Induction of CD4+ CD25+FOXP3+Lymphocytes:

Dendritic cells play an essential role in regulating the immune response by their ability to deliver either a priming or tolerogenic cell. In particular, these cells can induce regulatory T cells CD4+ CD25+ FOXP3+ that allow the maintenance of immunological tolerance and suppression of inflammatory immune responses. We therefore analyzed the effect of the treatment of monocytes by SIgA in the presence of IL-4 and GM-CSF on the induction of such cell population. Dendritic cells treated or not with SIgA were incubated with LPS (1 μg/ml) for 2 hours before to be washed and co-cultured with autologous CD3 T lymphocytes in a ratio of 1/5. After 6 days of culture the cells were analyzed by flow cytometry and FOXP3 mRNA quantified by real time PCR.

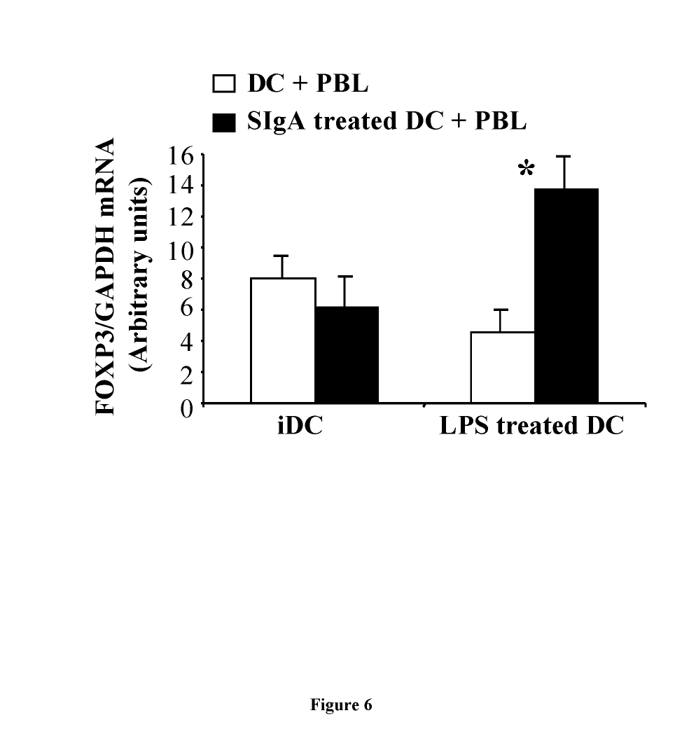

Our results show that dendritic cells that have been differentiated in the presence of SIgA induce a higher percent of T cells that are CD4high CD25high compared to untreated DC (22% vs 11.8)). Quantification of transcriptional factor Foxp3 by real-time PCR, after a normalization of results against a reporter gene encoding the GAPDH, shows 3 folds increase of Foxp3 in SIgA treated DCs compared to untreated cells (FIG. 6).

Induction of Tolerogenic Dendritic Cells Involves Mainly an Interaction of SIgA with the CD71 and Partially with CD89 Receptors Expressed on Dendritic Cells:

Monocytes express on their surface two major receptors of IgA namely CD89 (FcalphaRI) and the transferrin receptor (TfR/CD71). In order to determine the role of CD89 and CD71 in the interaction of SIgA with dendritic cells allowing to induction of tolerogenic profile, we incubated monocytes with either recombinant CD89 or CD71 before addition of SIgA during differentiation step. At day 7 of differentiation, the cells were stimulated with LPS and the expression of activation markers CD80 and CD83 was analyzed 24 hours later by flow cytometry.

Our results confirm that LPS, which signal through TLR4, do not induce a maturation of dendritic cells when differentiated in the presence of SIgA, as showed by expression of CD83 (0.4%) and CD80 (6%) compared to 33% and 46%, respectively, obtained for SIgA-untreated DCs. In contrast, LPS induces the maturation of dendritic cells differentiated in the presence of recombinant CD71 protein and SIgA. Indeed, the percentage of expression of the maturation markers CD80 and CD83 reached the level observed when dendritic cells were differentiated in the absence of treatment with SIgA (from 6% to 49% for CD80 and from 0.4% to 35% for CD83). A much more moderate but significant effect was observed when cells were pre-incubated with recombinant CD89 protein before addition of SIgA with an increase in the percent of expression of the maturation markers CD80 and CD83 from 6% to 18% and from 0.4% to 9%, respectively.

Altogether, these results show that the effect of SIgA leading to the induction of tolerogenic dendritic cells implies mainly the CD71 and partially the CD89 expressed on DCs. The tolerogenic property of SIgA-treated DCs could also result of a dual signal through both the CD71 and CD89. In contrast to the reported anti-inflammatory property of IgA, no tolerogenic effect has been reported for transferrin protein, the main ligand of CD71 receptor.

SIgA Treatment Induced an Inhibition of c-Rel Nuclear Translocation in DCs Stimulated with LPS:

NF-κB (nuclear factor kappa-light-chain-enhancer of activated B cells) is a protein complex that controls the transcription of DNA. NF-κB, that is involved in cellular responses to stimuli such as bacterial LPS, plays a key role in the regulation of the immune response and many genes involved in inflammation and autoimmunity. NF-κB is thus found to be chronically active in many inflammatory diseases, such as inflammatory bowel disease, arthritis and gastritis. We thus investigated the effect of SIgA treatment of DCs on the capability of LPS to induce nuclear translocation of NF-κB family main members (c-Rel, p50 and p65/RelA). We showed that SIgA treatment of DCs induced a deep inhibition of translocation to nucleus of c-Rel after LPS stimulation by comparison to SIgA untreated DCs. Furthermore, SIgA treatment of DCs inhibited only partially the translocation of p50 and p65/RelA to nucleus as shown in the.

Secretory IgA Blocks the Maturation of Dendritic Cells Differentiated from Mice Bone Marrow:

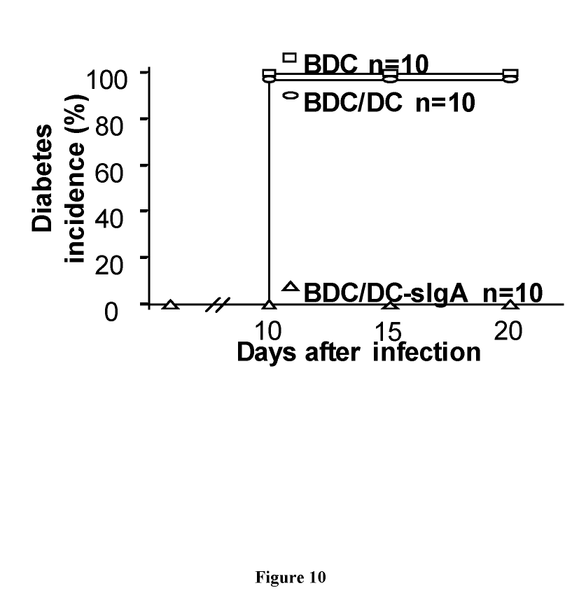

In vitro cultures of mouse bone marrow precursors with granulocyte/macrophage colony-stimulating factor (GM-CSF) induces the differentiation of DC which mimic both phenotypically and functionally circulating myeloid inflammatory CD11c⁺/CD11b⁺ DCs. Immature DCs generated in these conditions presents a phenotype and morphological aspects of DCs. LPS-maturation of bone marrow-derived DCs (BMDCs) induces the up-regulation of co-stimulatory and class II molecules (FIG. 10). We therefore added SIgA in GM-CSF cultures and differentiated DC in vitro. DCs primed with SIgA (DC-SIgA) were phenotypically and morphologically similar to classical DC. However SIgA-treated DC were not able to undergo LPS-dependent maturation.

Tolerogenic Potential of SIgA-Primed Mice DC:

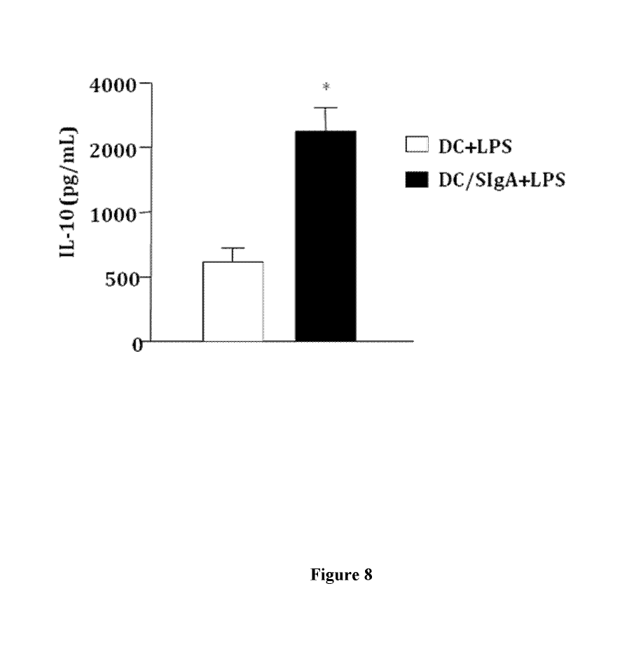

We observed that SIgA-conditioned DCs are unable to undergo LPS-induced maturation. There is a general consensus that whereas mature DC induces immunity, quiescent DC induces tolerance. The molecular mechanism implicated seems to be dependent on both co-stimulatory molecules and environmental cytokines which modulate immune functions. IL-10 is a well known cytokine suggested to be implicated in the induction of tolerogenic DCs. Therefore we looked whether SIgA-DC were able to secrete IL-10 after stimulation. As observed in FIG. 8, SIgA-DC secrete large amounts of IL-10. The induction of CD4⁺CD25⁺FOXP3⁺ regulatory T cells (Tregs) expansion/proliferation has been used to evaluate the tolerogenic potential of DCs. Since both TGF-β and IL-10 have been shown to be crucial in the induction of Tregs we co-cultured SIgA-DC and naïve monoclonal T cells specific for a pancreatic beta cell antigen (BDC) or specific for ovalbumine peptide (OTII) in the presence or in the absence of TGF-β and looked for the induction of Tregs. Whereas untreated-DCs where unable to generate Tregs, SIgA-DCs generated large amounts of those cells.

Therapeutic Potential of SIgA Treated DCs in Auto-Immune Diseases:

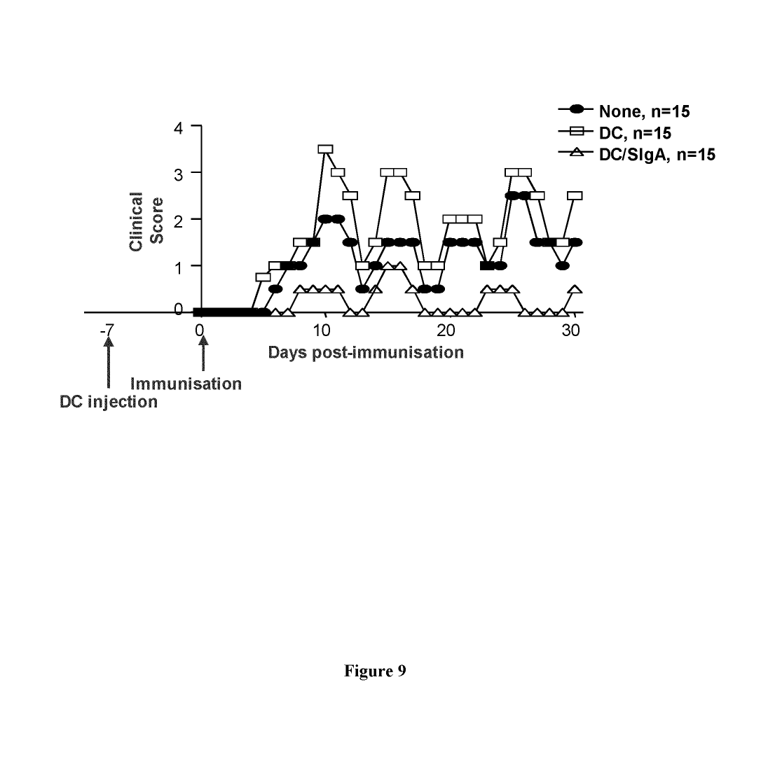

Our data propose that SIgA-conditioned DCs could induce tolerance. To test this hypothesis in vivo we use two models of auto-immune disease (type 1 diabetes and experimental auto-immune encephalomyelitis) to evaluate the therapeutic potential of SIgA-DCs. We have previously shown that MOG35-55-immunized NOD mice develop an experimental auto-immune encephalomyelitis (EAE) drove by an I-Ag7-restricted CD4+ T cells. Conventional DCs or SIgA-DCs were loaded with MOG35-55 peptide and injected 7 days before MOG35-55 immunization. Whereas conventional DCs aggravated EAE manifestations, SIgA-DCs renders NOD mice resistant to the induction of EAE (FIG. 9).

TABLE I
Clinical severity of EAE^{a}

| | | | | Mean | Mean | |
|---|---|---|---|---|---|---|
| | | | Mean Day | Maximum | Cumulative | |
| | | Incidence | of Onset^{b} | Severity | Severity | Mortality |
| Treatment | n | (%) | (SEM) | (SEM) | (SEM) | (%) |
| none | 15 | 100 | 7.8 +/− 0.7 | 2.3 +/− 0.2 | 42 +/− 15 | 6.7 |
| DC | 15 | 100 | 6.5 +/− 0.5 | 3.5 +/− 0.2* | 63 +/− 10 | 13.3 |
| DC/SIgA | 15 | 33 | 9.6 +/− 0.9** | 0.5 +/− 0.1*** | 11 +/− 4*** | 0 |

^{a}The pooled data from two independant experiments are presented. Statistical significance was tested compared to untreated mice.
*p < 0.05 calculated using the long-rank test.
^{b}Diseased animals only.

We also validated the therapeutic potential of SIgA-DCs to prevent diabetes induced by the transfer of diabetogenic T cells (BDC) in T cell deficient mice (NOD CαKo). In this model diabetes is induced 10 days after diabetogenic T cell transfer. Injection of DBC/SIgA-DCs co-cultures completely prevented diabetes. However injection of DBC/DCs cultures induced diabetes development in all mice transferred 10 day after transfer (FIG. 10). Interestingly we found that Tregs frequencies were increased in pancreatic (PLN) and mesenteric lymph nodes (MLN) but not in spleen. Intracellular staining for IFN-γ and IL-10 show that over than 50% of cells infiltrating PLN were IFN-g⁻ IL-10⁺ T cells, whereas the transfer of BDC cultured in presence of untreated DCs lead to the expansion of IFN-g⁺ IL-10⁻ T cells in PLN and MLN. Altogether our data confirm the tolerogenic potential of SIgA-DCs and their capacity to prevent auto-immune diseases.

### REFERENCES

Throughout this application, various references describe the state of the art to which this invention pertains. The disclosures of these references are hereby incorporated by reference into the present disclosure.
1. Banchereau, J., and V. Pascual. 2006. Type I interferon in systemic lupus erythematosus and other autoimmune diseases. Immunity 25:383-92.
2. Banchereau, J., V. Pascual, and A. K. Palucka. 2004. Autoimmunity through cytokine-induced dendritic cell activation. Immunity 20:539-50.
3. Brandtzaeg, P. 1995. Molecular and cellular aspects of the secretory immunoglobulin system. Apmis 103:1-19.
4. Corthesy, B. 2007. Roundtrip ticket for secretory IgA: role in mucosal homeostasis? J Immunol 178:27-32.
5. Fagarasan, S., and T. Honjo. 2003. Intestinal IgA synthesis: regulation of front-line body defences. Nat Rev Immunol 3:63-72.
6. Favre, L., F. Spertini, and B. Corthesy. 2005. Secretory IgA possesses intrinsic modulatory properties stimulating mucosal and systemic immune responses. J Immunol 175:2793-800.
7. Heystek, H. C., C. Moulon, A. M. Woltman, P. Garonne, and C. van Kooten. 2002. Human immature dendritic cells efficiently bind and take up secretory IgA without the induction of maturation. J Immunol 168:102-7.
8. Jacob, C. M., A. C. Pastorino, K. Fahl, M. Carneiro-Sampaio, and R. C. Monteiro. 2008. Autoimmunity in IgA deficiency: revisiting the role of IgA as a silent housekeeper. J Clin Immunol 28 Suppl 1:S56-61.
9. Kanamaru, Y., U. Blank, and R. C. Monteiro. 2007. IgA Fc receptor I is a molecular switch that determines IgA activating or inhibitory functions. Contrib Nephrol 157:148-52.
10. Kanazawa, N. 2007. Dendritic cell immunoreceptors: C-type lectin receptors for pattern-recognition and signaling on antigen-presenting cells. J Dermatol Sci 45:77-86.
11. Kelsall, B. L., and F. Leon. 2005. Involvement of intestinal dendritic cells in oral tolerance, immunity to pathogens, and inflammatory bowel disease. Immunol Rev 206:132-48.
12. Kumar, H., T. Kawai, and S. Akira. 2009. Pathogen recognition in the innate immune response. Biochem J 420:1-16.
13. Macpherson, A. J., D. Gatto, E. Sainsbury, G. R. Harriman, H. Hengartner, and R. M. Zinkernagel. 2000. A primitive T cell-independent mechanism of intestinal mucosal IgA responses to commensal bacteria. Science 288:2222-6.
14. Macpherson, A. J., K. D. McCoy, F. E. Johansen, and P. Brandtzaeg. 2008. The immune geography of IgA induction and function. Mucosal Immunol 1:11-22.
15. Monteiro, R. C., and J. G. Van De Winkel. 2003. IgA Fc receptors. Annu Rev Immunol 21:177-204.
16. Moser, M. 2003. Dendritic cells in immunity and tolerance-do they display opposite functions? Immunity 19:5-8.
17. Naik, S. H., D. Metcalf, A. van Nieuwenhuijze, I. Wicks, L. Wu, M. O'Keeffe, and K. Shortman. 2006. Intrasplenic steady-state dendritic cell precursors that are distinct from monocytes. Nat Immunol 7:663-71.
18. Nimmerjahn, F., and J. V. Ravetch. 2008. Anti-inflammatory actions of intravenous immunoglobulin. Annu Rev Immunol 26:513-33.
19. Pasquier, B., P. Launay, Y. Kanamaru, I. C. Moura, S. Pfirsch, C. Ruffie, D. Henin, M. Benhamou, M. Pretolani, U. Blank, and R. C. Monteiro. 2005. Identification of FcalphaRI as an inhibitory receptor that controls inflammation: dual role of FcRgamma ITAM. Immunity 22:31-42.
20. Quan, C. P., A. Berneman, R. Pires, S. Avrameas, and J. P. Bouvet. 1997. Natural polyreactive secretory immunoglobulin A autoantibodies as a possible barrier to infection in humans. Infect Immun 65:3997-4004.
21. Rey, J., N. Garin, F. Spertini, and B. Corthesy. 2004. Targeting of secretory IgA to Peyer's patch dendritic and T cells after transport by intestinal M cells. J Immunol 172:3026-33.
22. Shortman, K., and S. H. Naik. 2007. Steady-state and inflammatory dendritic-cell development. Nat Rev Immunol 7:19-30.
23. Steinbrink, K., K. Mahnke, S. Grabbe, A. H. Enk, and H. Jonuleit. 2009. Myeloid dendritic cell: From sentinel of immunity to key player of peripheral tolerance? Hum Immunol.
24. Stoel, M., H. Q. Jiang, C. C. van Diemen, J. C. Bun, P. M. Dammers, M. C. Thurnheer, F. G. Kroese, J. J. Cebra, and N. A. Bos. 2005. Restricted IgA repertoire in both B-1 and B-2 cell-derived gut plasmablasts. J Immunol 174:1046-54.
25. Varol, C., L. Landsman, D. K. Fogg, L. Greenshtein, B. Gildor, R. Margalit, V. Kalchenko, F. Geissmann, and S. Jung. 2007. Monocytes give rise to mucosal, but not splenic, conventional dendritic cells. J Exp Med 204:171-80.
26. Varol, C., A. Vallon-Eberhard, E. Elinav, T. Aychek, Y. Shapira, H. Luche, H. J. Fehling, W. D. Hardt, G. Shakhar, and S. Jung. 2009. Intestinal lamina propria dendritic cell subsets have different origin and functions. Immunity 31:502-12.
27. Yrlid, U., C. D. Jenkins, and G. G. MacPherson. 2006. Relationships between distinct blood monocyte subsets and migrating intestinal lymph dendritic cells in vivo under steady-state conditions. J Immunol 176:4155-62.

1. A culture medium suitable for inducing dendritic cell differentiation comprising an effective amount of secretory immunoglobulins A (SIgA).

2. The culture medium according to claim 1, wherein said medium comprises granulocyte-macrophage colony-stimulating factor (GM-CSF) and interleukin-4 (IL-4).

3. The culture medium according to claim 1, wherein said SIgA is human SIgA.

4. A method for obtaining a population of tolerogenic dendritic cells comprising a step of culturing monocytes with culture medium comprising an effective amount of secretory immunoglobulins A (SIgA).

5. The method according to claim 4, wherein the monocytes are human monocytes.

6. A population of tolerogenic dendritic cells obtained by
culturing monocytes with culture medium comprising an effective amount of secretory immunoglobulins A (SIgA).

7. A pharmaceutical composition comprising a population of tolerogenic dendritic cells obtained by culturing monocytes with culture medium comprising an effective amount of secretory immunoglobulins A (SIgA), and a pharmaceutically acceptable carrier or excipient.

8-10. (canceled)

11. A method for treating an autoimmune or inflammatory disease comprising the step of administering to a patient in need thereof a pharmaceutically effective amount of the population of tolerogenic dendritic cells obtained by
culturing monocytes with culture medium comprising an effective amount of secretory immunoglobulins A (SIgA).

12. A method for inducing transplant tolerance comprising the step of administering to a patient in need thereof a pharmaceutically effective amount of a population of tolerogenic dendritic cells obtained by
culturing monocytes with culture medium comprising an effective amount of secretory immunoglobulins A (SIgA).

13. (canceled)

14. The method of claim 11, wherein the autoimmune or inflammatory disease is selected from the group consisting of allergy, multiple sclerosis, auto-immune type 1 diabetes (diabetes mellitus), rheumatoid arthritis, inflammatory bowel disease, systemic lupus erythematosus, Graves' disease and nephropathy, vasculitides, scleroderma, psoriasis, autoimmune thyroid disease, glomerulonephritis and Sjogren's disease.
